(19) United States
(12) Patent Application Publication
(10) Pub. No.: US 2023/0052238 A1
(43) Pub. Date: Feb. 16, 2023

(54) Unisex Urine Collection Assembly

(71) Applicant: Olubisi Oluwasogo, Fremont, CA (US)

(72) Inventor: Olubisi Oluwasogo, Fremont, CA (US)

(21) Appl. No.: 17/399,766

(22) Filed: Aug. 11, 2021

(51) Int. Cl. A61F 5/453; A61M 1/00; A61F 5/455

(52) U.S. Cl. CPC A61F 5/453; A61F 5/4553; A61M 1/87; A61G 9/00

(57) ABSTRACT

A unisex urine collection assembly for use by either sex in a non-ambulatory setting includes a tube, which has a cutout positioned in a top face thereof and extending from proximate to a midpoint toward a first end thereof. A second end of the tube is closed. A connector engaged to the first end of the tube can engage a conduit extending from a suctioning unit. Each of a pair of pads is engaged to the tube and extends from a respective opposed edge of the cutout thereinto so that the pads defines a slot. The pads are resiliently flexible and can sealably engage skin of the user. The pads can be positioned between labia of a female user or to bracket a penis inserted into the slot by a male user. Urine expelled by the user is suctioned from the tube into a reservoir.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### Field of the Invention

The disclosure relates to urine collection assemblies and more particularly pertains to a new urine collection assembly for use by either sex in a non-ambulatory setting.

### Description of Related Art Including Information Disclosed Under 37 CFR 1.97 And 1.98.

The prior art relates to urine collection assemblies, which may comprise receptacles for inserting genitalia, drain tubes connected to bags, portable urinals, and the like. What is lacking in the prior art is a unisex urine collection assembly wherein the assembly comprises pads lining a cutout in a tube. The pads are configured to be insertable between labia of a female user or to bracket a penis of a male user, such that the pads sealably engage skin of the user when suction is applied to the tube.

### BRIEF SUMMARY OF THE INVENTION

An embodiment of the disclosure meets the needs presented above by generally comprising a tube, a connector, and a pair of pads. The tube has a cutout positioned in a top face thereof. The cutout extends from proximate to a midpoint of the tube toward a first end thereof. The tube has a second end, which is closed.

The connector is engaged to the first end of the tube and is configured to engage a conduit extending from a suctioning unit so that the tube is in fluidic communication with the suctioning unit.

Each pad is engaged to the tube and extends from a respective opposed edge of the cutout thereinto so that the pair of pads defines a slot. The pads are resiliently flexible and thus configured to sealably engage skin of a user when suction is applied by the suctioning unit. The pads are configured to be positioned between labia of a female user or to bracket a penis inserted into the slot by a male user. Urine expelled by the user is suctioned from the tube into a reservoir by action of the suctioning unit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWINGS

The disclosure will be better understood and objects other than those set forth above will become apparent when consideration is given to the following detailed description thereof. Such description makes reference to the annexed drawings wherein:

FIG. 1 is an isometric perspective view of a unisex urine collection assembly according to an embodiment of the disclosure.

FIG. 2 is a top view of an embodiment of the disclosure.

FIG. 3 is a bottom view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

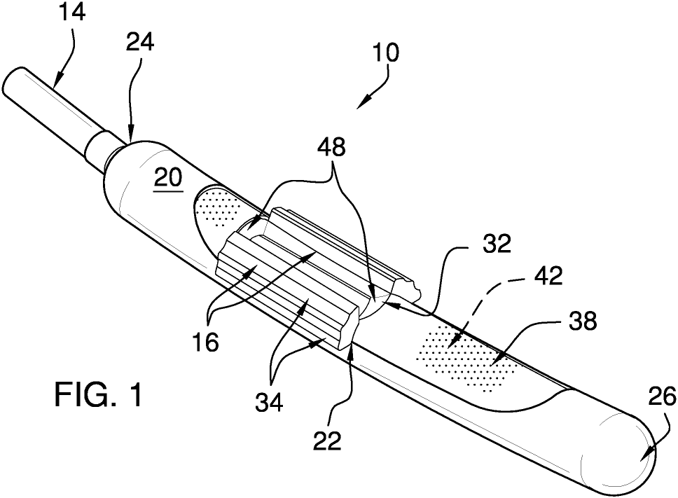

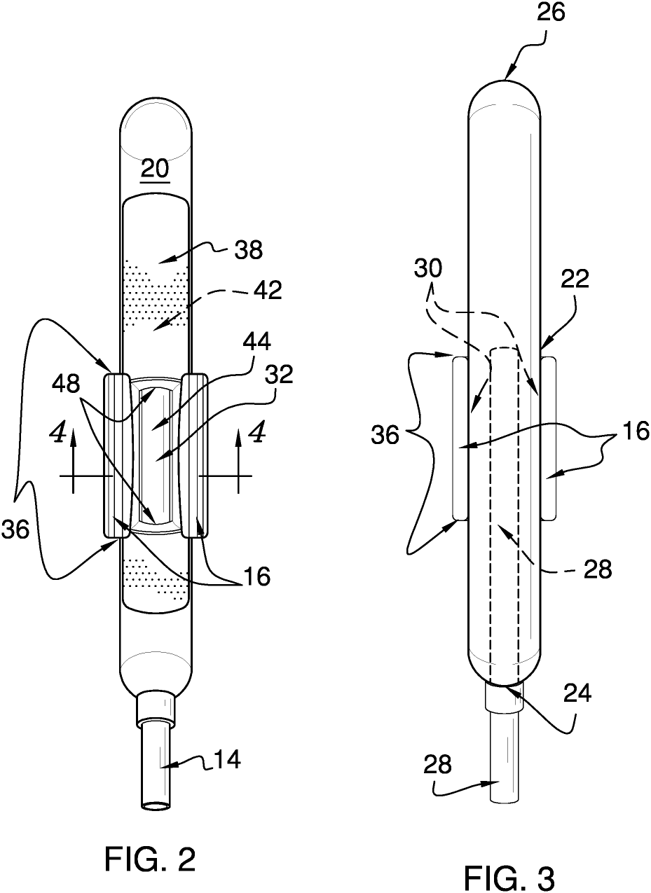

With reference now to the drawings, and in particular to FIGS. 1 through 5 thereof, a new urine collection assembly embodying the principles and concepts of an embodiment of the disclosure and generally designated by the reference numeral 10 will be described.

As best illustrated in FIGS. 1 through 5, the unisex urine collection assembly 10 generally comprises a tube 12, a connector 14, and a pair of pads 16. The tube 12 has a cutout 18 positioned in a top face 20 thereof. The cutout 18 extends from proximate to a midpoint 22 of the tube 12 toward a first end 24 thereof. The tube 12 has a second end 26, which is closed. The second end 26 may be arcuate, as shown in FIGS. 1-3, to increase a comfort level for a user.

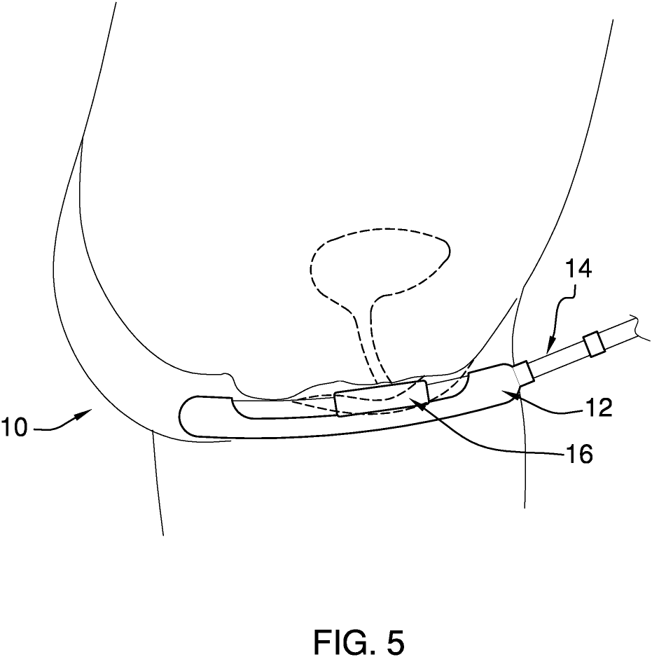

The connector 14 is engaged to the first end 24 of the tube 12 and is configured to engage a conduit extending from a suctioning unit (as shown in FIG. 5, suctioning unit not shown) so that the tube 12 is in fluidic communication with the suctioning unit. The connector 14 comprises a pipe 28, which extends into the tube 12 to proximate to the midpoint 22 of the tube 12. The pipe 28 comprises plastic so that the pipe 28 is resiliently flexible. The pipe 28 being flexible allows for bending to an optimal angle, relative to the tube 12, for connection to the conduit extending from the suctioning unit.

Each pad 16 is engaged to the tube 12 and extends from a respective opposed edge 30 of the cutout 18 thereinto so that the pair of pads 16 defines a slot 32. The pads 16 are resiliently flexible and thus configured to sealably engage skin of the user when suction is applied by the suctioning unit. The pads 16 are configured to be positioned between labia of a female user, as shown in FIG. 5, or to bracket a penis inserted into the slot 32 by a male user (not shown). Urine expelled by the user is suctioned from the tube 12 into a reservoir (not shown) by action of the suctioning unit. The assembly 10 is anticipated to be of particular use for non-ambulatory users.

The pads 16 comprise foamed elastomer, rubber, or silicone. The pads 16 have a plurality of indentations 34 positioned therein, with each indentation 34 extending between opposed ends 36 of a respective pad 16. The indentations are configured to promote sealing of the pad 16 to the skin of the user.

In one embodiment, the cutout 18 extends bidirectionally from the midpoint 22 of the tube 12. A panel 38 is engaged to an inner surface 40 of the tube 12 and extends circumferentially therearound so that the cutout 18 is covered by the panel 38. The panel 38 is configured to wick so that urine contacting the panel 38 is wicked into the tube 12. The panel 38 comprises silk, wool, polyester, polyethylene, or microfiber cloth.

The pads 16 are engaged to the panel 38 and extend from the midpoint 22 of the tube 12 toward the first end 24 of the tube 12. The slot 32 that is defined by the pads 16 extends through the panel 38.

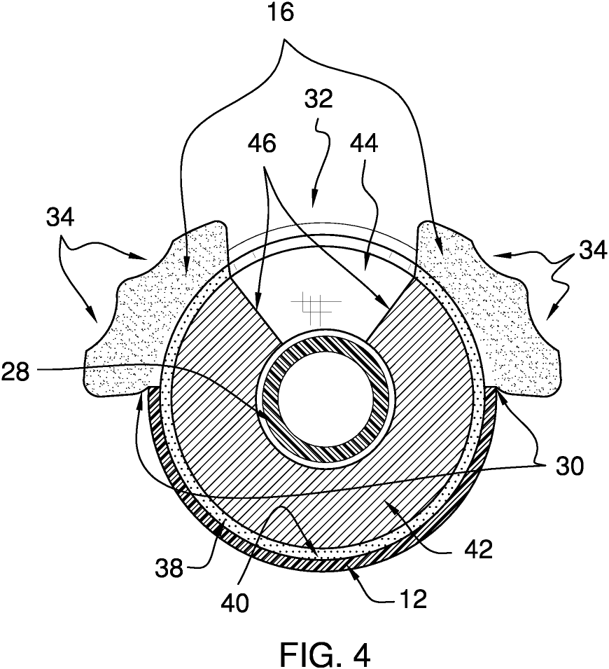

A filler 42 is positioned in the tube 12 and is engaged to the panel 38. The filler 42 is permeable so that urine passes therethrough. The filler 42 comprises sponge, cotton, or gauze. The filler 42 has a void 44 positioned therein and extending from the slot 32 into the tube 12, as shown in FIG. 4. The void 44 is configured for insertion of genitalia of the user. The present invention also anticipates the panel 38 lining opposed sides 46 and opposed ends 48 of the void 44.

In use, the pads 16 are positioned between the labia of the female user or to bracket the penis inserted into the slot 32 by the male user. The suctioning unit is engaged to create a partial vacuum within the tube 12. The pads 16, being resiliently flexible, sealably engage the skin of the user to couple the assembly 10 to the user. Urine expelled by the user is suctioned from the tube 12 into the reservoir by action of the suctioning unit.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A unisex urine collection assembly comprising:
a tube having a cutout positioned in a top face thereof, the cutout extending from proximate to a midpoint of the tube toward a first end thereof, the tube having a second end, the second end being closed;
a connector engaged to the first end of the tube and being configured for engaging a conduit extending from a suctioning unit, such that the tube is in fluidic communication with the suctioning unit; and
a pair of pads, each pad being engaged to the tube such that the pad extends from a respective opposed edge of the cutout thereinto, such that the pair of pads defines a slot, the pads being resiliently flexible, such that the pads are configured for sealably engaging skin of a user when suction is applied by the suctioning unit, wherein the pads are configured for positioning between labia of a female user or for bracketing a penis inserted into the slot by a male user, such that urine expelled by the user is suctioned from the tube into a reservoir by action of the suctioning unit.

2. The unisex urine collection assembly of claim 1, wherein the second end is arcuate.

3. The unisex urine collection assembly of claim 1, wherein the connector comprises a pipe.

4. The unisex urine collection assembly of claim 3, wherein the pipe comprises plastic such that the pipe is resiliently flexible.

5. The unisex urine collection assembly of claim 3, wherein the pipe extends into the tube to proximate to the midpoint of the tube.

6. The unisex urine collection assembly of claim 1, wherein the pads comprise foamed elastomer, rubber, or silicone.

7. The unisex urine collection assembly of claim 1, wherein the pads have a plurality of indentations positioned therein, each indentation extending between opposed ends of a respective pad, wherein the indentations are configured for promoting sealing of the pad to the skin of the user.

8. The unisex urine collection assembly of claim 1, further including:
the cutout extending bidirectionally from the midpoint of the tube;
a panel engaged to an inner surface of the tube and extending circumferentially therearound, such that the cutout is covered by the panel, the panel being configured for wicking, such that urine contacting the panel is wicked into the tube; and
the pads being engaged to the panel and extending from the midpoint of the tube toward the first end of the tube, the slot defined by the pads extending through the panel.

9. The unisex urine collection assembly of claim 8, wherein the panel comprises silk, wool, polyester, polyethylene, or microfiber cloth.

10. The unisex urine collection assembly of claim 8, further including a filler positioned in the tube and engaged to the panel, the filler being permeable such that urine passes therethrough, the filler having a void positioned therein and extending from the slot into the tube, wherein the void is configured for insertion of genitalia of the user.

11. The unisex urine collection assembly of claim 10, wherein the filler comprises sponge, cotton, or gauze.

12. A unisex urine collection assembly comprising:
a tube having a cutout positioned in a top face thereof, the cutout extending from proximate to a midpoint of the tube toward a first end thereof, the tube having a second end, the second end being closed, the second end being arcuate;
a connector engaged to the first end of the tube and being configured for engaging a conduit extending from a suctioning unit, such that the tube is in fluidic communication with the suctioning unit, the connector comprising a pipe, the pipe comprising plastic such that the pipe is resiliently flexible, the pipe extending into the tube to proximate to the midpoint of the tube;
a pair of pads, each pad being engaged to the tube such that the pad extends from a respective opposed edge of the cutout thereinto, such that the pair of pads defines a slot, the pads being resiliently flexible, such that the pads are configured for sealably engaging skin of a user when suction is applied by the suctioning unit, wherein the pads are configured for positioning between labia of a female user or for bracketing a penis inserted into the slot by a male user, such that urine expelled by the user is suctioned from the tube into a reservoir by action of the suctioning unit, the pads comprising foamed elastomer, rubber, or silicone, the pads having a plurality of indentations positioned therein, each indentation extending between opposed ends of a respective pad, wherein the indentations are configured for promoting sealing of the pad to the skin of the user;
the cutout extending bidirectionally from the midpoint of the tube;
a panel engaged to an inner surface of the tube and extending circumferentially therearound, such that the cutout is covered by the panel, the panel being configured for wicking, such that urine contacting the panel is wicked into the tube, the panel comprising silk, wool, polyester, polyethylene, or microfiber cloth;
the pads being engaged to the panel and extending from the midpoint of the tube toward the first end of the tube, the slot defined by the pads extending through the panel; and
a filler positioned in the tube and engaged to the panel, the filler being permeable such that urine passes therethrough, the filler having a void positioned therein and extending from the slot into the tube, wherein the void is configured for insertion of genitalia of the user, the filler comprising sponge, cotton, or gauze.
